(19) United States
(12) Patent Application Publication
(10) Pub. No.: US 2025/0388534 A1
(43) Pub. Date: Dec. 25, 2025

(54) SUPRAMOLECULAR TRANEXAMIC ACID-MANDELIC ACID IONIC SALT AS WELL AS PREPARATION METHOD THEREFOR AND USE THEREOF

(71) Applicant: SHENZHEN SHINEHIGH INNOVATION CO., LTD., Nanshan Dist. (CN)

(72) Inventors: Zhenyuan Wang, Shenzhen (CN); Jiaheng Zhang, Shenzhen (CN); Nuo Li, Shenzhen (CN); Junguang Liu, Shenzhen (CN); Li Zhou, Shenzhen (CN); Yuanzhi Yao, Shenzhen (CN); Jing Su, Shenzhen (CN)

(73) Assignee: SHENZHEN SHINEHIGH INNOVATION CO., LTD., Nanshan Dist. (CN)

(21) Appl. No.: 19/310,818

(22) Filed: Aug. 26, 2025

Related U.S. Application Data
(63) Continuation-in-part of application No. PCT/CN2023/135958, filed on Dec. 1, 2023.

(30) Foreign Application Priority Data
Dec. 7, 2022 (CN) 202211560438.6

(51) Int. Cl. C07C 291/04; A61K 8/41; A61K 31/205; A61P 29/00; A61P 39/06; A61Q 19/00

(52) U.S. Cl. CPC C07C 291/04; A61K 8/416; A61K 31/205; A61P 29/00; A61P 39/06; A61Q 19/00; C07C 2601/14

(57) ABSTRACT

Provided are a supramolecular tranexamic acid-mandelic acid ionic salt as well as a preparation method therefor and a use thereof, relating to the technical field of pharmaceutical and cosmetic compounds. The supramolecular tranexamic acid-mandelic acid ionic salt has a structural formula as shown in formula I. Tranexamic acid and mandelic acid undergo reaction and bonding to form a supramolecular salt. The skin permeability of mandelic acid can be effectively improved and less irritation is caused to the skin while the efficacy of mandelic acid is well maintained.

### CROSS-REFERENCE TO RELATED APPLICATIONS

This is a Continuation-in-Part of PCT Application No. PCT/CN2023/135958 filed Dec. 1, 2023, which in turn claims the benefit of priority of Chinese Patent Application No. 202211560438.6, filed with the China National Intellectual Property Administration on Dec. 7, 2022, and entitled “SUPRAMOLECULAR TRANEXAMIC ACID-MANDELIC ACID IONIC SALT AS WELL AS PREPARATION METHOD THEREFOR AND USE THEREOF.” Both applications are incorporated by reference herein in their entireties.

### TECHNICAL FIELD

The present application relates to the field of compound technology for pharmaceuticals and cosmetics, and specifically to a supramolecular tranexamic acid-mandelic acid ionic salt, and a preparation method therefor and use thereof.

### BACKGROUND

Mandelic acid (MA) has a chemical structure of a-hydroxyphenylacetic acid. It is an important chiral pharmaceutical intermediate and fine chemical product, serving as the raw material for synthesizing cyclandelate for vasodilators, mandelic acid urotropin for anti-inflammatory medicines against urinary tract infection, mandelic acid benzyl ester for antispasmodic agents, and the like.

Transdermal administration is currently a common method for the use of mandelic acid products such as transdermal administration to treat inflammation, but conventional topical formulations of mandelic acid have difficulty penetrating the stratum corneum barrier, resulting in low bioavailability and limited application of mandelic acid.

### SUMMARY

The purpose of the present application is to provide a supramolecular tranexamic acid-mandelic acid ionic salt, and a preparation method therefor and use thereof, which can effectively improve the skin permeability of mandelic acid while maintaining efficacy and causing low skin irritation.

Embodiments of the present application are implemented as follows:

In a first aspect, the embodiments of the present application provide a supramolecular tranexamic acid-mandelic acid ionic salt, which have a structural formula as shown in Formula 1:

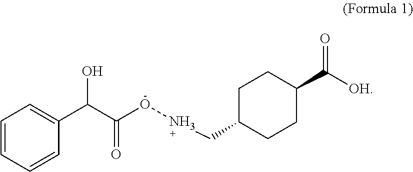

In a second aspect, the embodiments of the present application provide a preparation method for supramolecular tranexamic acid-mandelic acid ionic salt as provided in the embodiments of the first aspect, which includes reacting tranexamic acid and mandelic acid to obtain the supramolecular tranexamic acid-mandelic acid ionic salt shown in Formula 1.

In a third aspect, the embodiments of the present application provide use of supramolecular tranexamic acid-mandelic acid ionic salt as a raw material in the preparation of pharmaceuticals or cosmetics as provided in the embodiments of the first aspect.

The beneficial effects of the supramolecular tranexamic acid-mandelic acid ionic salt, and preparation method therefor and use thereof provided in the embodiments of the present application include: in the present application, the supramolecular tranexamic acid-mandelic acid ionic salt shown in Formula 1 is obtained by using tranexamic acid and mandelic acid as precursors through ionization to form a salt. The supramolecular tranexamic acid-mandelic acid ionic salt can better maintain the efficacy of mandelic acid, such as anti-oxidation, inhibition of melanocyte activity, and inhibition of tyrosinase activity; at the same time, it can effectively improve the skin permeability of mandelic acid and improve the bioavailability of mandelic acid; moreover, it has low skin irritation, which enhances the application effect.

### BRIEF DESCRIPTION OF DRAWINGS

In order to more explicitly illustrate the technical solution of the embodiments of the present application, the drawings required for the embodiments are briefly described below. It should be understood that the following drawings only show some embodiments of the present application and should not be construed as limiting the scope of the present application. A person of ordinary skill in the art can derive other related drawings based on these drawings without the exercise of inventive efforts.

FIG. 1 is a flow chart of a preparation method for a supramolecular tranexamic acid-mandelic acid ionic salt according to an embodiment of the present application;

FIG. 2 is a thermogravimetric analysis curve of supramolecular tranexamic acid-mandelic acid ionic salt, tranexamic acid monomer, and mandelic acid monomer according to Embodiment 1 of the present application;

FIG. 3 is an ¹H-NMR spectrum of a supramolecular tranexamic acid-mandelic acid ionic salt according to Embodiment 1 of the present application;

FIG. 4 is a schematic diagram of a molecular structure of a supramolecular tranexamic acid-mandelic acid ionic salt crystal according to Embodiment 5 of the present application;

FIG. 5 is a statistical diagram of the penetration efficiency according to Test Example 1 of the present application;

FIG. 6 is a line graph of DPPH⋅ radical scavenging rate according to Test Example 4 of the present application; and

FIG. 7 is a line graph of ABTS⁺⋅. scavenging rate according to Test Example 4 of the present application.

### DETAILED DESCRIPTION OF THE EMBODIMENTS

The technical solution of the present application is described in detail below in conjunction with specific embodiments. Unless otherwise specified, conditions not explicitly stated in the embodiments shall be understood as conventional conditions or conditions recommended by manufacturers. Reagents or instruments not specifically identified by manufacturer are all commercially available conventional products.

In a first aspect, the embodiments of the present application provide a supramolecular tranexamic acid-mandelic acid ionic salt, which have a structural formula as shown in Formula 1;

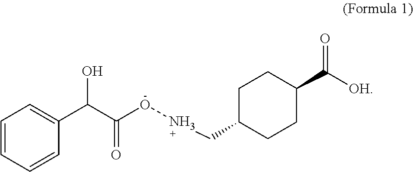

In some possible embodiments, the supramolecular tranexamic acid-mandelic acid ionic salt includes a tranexamic acid structure and a mandelic acid structure with a mole ratio (that is, a ratio of the amount of substances) of 1:5 to 5:1. In the embodiment, the tranexamic acid structure and the mandelic acid structure have a suitable ratio of substances, so that the supramolecular tranexamic acid-mandelic acid ionic salt has a better permeation effect.

As an example, the mole ratio of the tranexamic acid structure and the mandelic acid structure is, for example, but not limited to, any one of the following point values: 1:5, 1:4, 1:3, 1:2, 1:1, 2:5, 2:3, 2:1, 3:5, 3:4, 3:2, 3:1, 4:5, 4:3, 4:1, 5:4, 5:3, 5:2, and 5:1, or a range value between any two.

In a second aspect, the embodiments of the present application provide a preparation method for supramolecular tranexamic acid-mandelic acid ionic salt as provided in the embodiments of the first aspect, which includes reacting tranexamic acid and mandelic acid to obtain the supramolecular tranexamic acid-mandelic acid ionic salt shown in Formula 1.

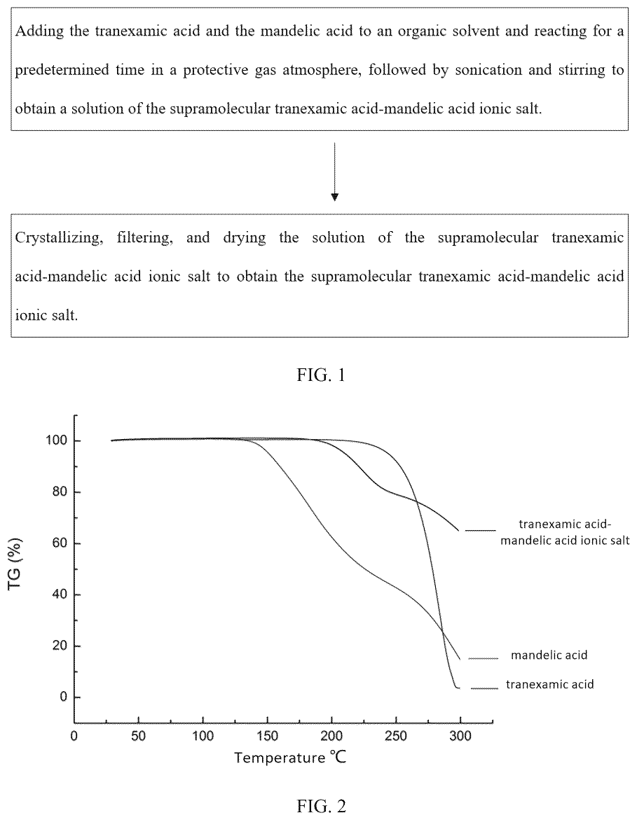

Referring to FIG. 1, as an example, the step of reacting the tranexamic acid and the mandelic acid to obtain the supramolecular tranexamic acid-mandelic acid ionic salt as shown in Formula 1 includes the following operations: adding the tranexamic acid and the mandelic acid to an organic solvent and reacting for a predetermined time in a protective gas atmosphere, followed by sonication and stirring to obtain a solution of the supramolecular tranexamic acid-mandelic acid ionic salt; and crystallizing, filtering, and drying the solution of the supramolecular tranexamic acid-mandelic acid ionic salt to obtain the supramolecular tranexamic acid-mandelic acid ionic salt. Specifically, the protective gas atmosphere refers to an anti-oxidation protective atmosphere, which is, for example, an atmosphere including one or more inert gases such as helium, argon, nitrogen, and carbon dioxide.

The dosage ratio of the tranexamic acid and the mandelic acid can refer to the mole ratio of the tranexamic acid structure to the mandelic acid structure in the supramolecular tranexamic acid-mandelic acid ionic salt, that is, as an example, the mole ratio of the tranexamic acid to the mandelic acid is 1:5 to 5:1.

The type of organic solvent is not limited as long as it can better achieve the dissolution and dispersion of the tranexamic acid and the mandelic acid. As an example, the organic solvent includes one or more of acetonitrile, ethanol, and methanol.

In order to make the salt formation reaction through ionization of the tranexamic acid and the mandelic acid fully carried out, the predetermined reaction time may optionally range from 12 h to 48 h. The predetermined time of the salt formation reaction through ionization is, for example, but not limited to, any one of the following point values: 12 h, 18 h, 24 h, 30 h, 36 h, 42 h, and 48 h, or a range value between any two.

In order to achieve a better ultrasonic effect, optionally, during the ultrasonic process, at least one of the following conditions (a1) to (a5) is satisfied. (a1) The temperature of the ultrasonic field is 50° C. to 90° C., for example, but not limited to, any one of the following point values: 50° C., 60° C., 70° C., 80° C., and 90° C., or a range value between any two. (a2) The ultrasonic frequency is 20 kHz to 60 kHz, for example, but not limited to, any one of the following point values: 20 kHz, 30 kHz, 40 kHz, 50 kHz, and 60 kHz, or a range value between any two. (a3) The ultrasonic power is 700 W to 6000 W, for example, but not limited to, any one of the following point values: 700 W, 1000 W, 1500 W, 2000 W, 2500 W, 3000 W, 3500 W, 4000 W, 4500 W, 5000 W, 5500 W, and 6000 W, or a range value between any two. (a4) The ultrasonic time is 6 h to 12 h, for example, but not limited to, any one of the following point values: 6 h, 7 h, 8 h, 9 h, 10 h, 11 h, and 12 h, or a range value between any two. (a5) Ultrasound for 2 s to 10 s at every interval of 1 s to 5 s. Specifically, the time of the ultrasonic interval is, for example, but not limited to, any one of the following point values: 1 s, 2 s, 3 s, 4 s, and 5 s, or a range value between any two, and the ultrasonic time between two intervals is, for example, but not limited to, any one of the following point values: 2 s, 3 s, 4 s, 5 s, 6 s, 7 s, 8 s, 9 s, and 10 s, or a range value between any two.

In order to achieve a better stirring effect, optionally, during the stirring process, the following conditions (b1) and/or (b2) are satisfied.

(b1) The stirring rate is 30 rad/min to 250 rad/min, for example, but not limited to, any one of the following point values: 30 rad/min, 50 rad/min, 100 rad/min, 150 rad/min, 200 rad/min, and 250 rad/min, or a range value between any two. (b2) The stirring time is 12 h to 48 h, for example, but not limited to, any one of the following point values: 12 h, 18 h, 24 h, 30 h, 36 h, 42 h, and 48 h, or a range value between any two.

In the present application, the crystallization method is not limited as long as it can effectively crystallize and separate the supramolecular tranexamic acid-mandelic acid ionic salt obtained by the reaction from the salt solution. Optionally, during the crystallization process, the following conditions (c1) and/or (c2) are satisfied.

(c1) Concentration crystallization is performed under vacuum conditions. (c2) Cooling crystallization is performed under a temperature condition of 5° C. to 15° C. Specifically, the cooling crystallization temperature is, for example, but not limited to, any one of the following point values: 5° C., 8° C., 10° C., 12° C., and 15° C., or a range value between any two.

In order to achieve efficient drying while avoiding the destruction of the supramolecular tranexamic acid-mandelic acid ionic salt, optionally, the drying temperature is 50° C. to 90° C., for example, but not limited to, any one of the following point values: 50° C., 60° C., 70° C., 80° C., and 90° C., or a range value between any two. The drying time depends on the degree of drying and can be selected as 36 h to 60 h, for example, but not limited to, any one of the following point values: 36 h, 42 h, 48 h, 54 h, and 60 h, or a range value between any two.

In a third aspect, the embodiments of the present application provide use of supramolecular tranexamic acid-mandelic acid ionic salt as a raw material in the preparation of pharmaceuticals or cosmetics as provided in the embodiments of the first aspect.

As an example, pharmaceuticals and cosmetics include, but are not limited to, products that achieve one or more of the following target functions, including: anti-oxidation, inhibition of melanocyte activity, inhibition of tyrosinase activity, DPPH⋅ radical scavenging, ABTS+⋅ scavenging, and anti-inflammatory.

The technical solution of the present application is described below in conjunction with specific embodiments and test examples.

### I. EXAMPLES AND COMPARATIVE EXAMPLES

### Embodiment 1

A supramolecular tranexamic acid-mandelic acid ionic salt, the preparation method of which is as follows.

In an inert gas atmosphere, 0.10 mol of tranexamic acid and 0.10 mol of mandelic acid are added to acetonitrile for 24 h; under the condition of 75° C., the ultrasonic frequency is 40 kHz, the ultrasonic power is 2000 W, the ultrasonic time is 12 h, and the interval time is 10 s every 3 s; and the stirring time is 24 h and the stirring rate is 60 rad/min to obtain a solution of supramolecular tranexamic acid-mandelic acid ionic salt. Under vacuum conditions, the obtained solution of supramolecular tranexamic acid-mandelic acid ionic salt is concentrated and crystallized; then, dried in a vacuum drying oven for 48 h at a drying temperature of 60° C. to obtain supramolecular tranexamic acid-mandelic acid ionic salt.

### Embodiment 2

A supramolecular tranexamic acid-mandelic acid ionic salt, the preparation method of which is as follows. In an inert gas atmosphere, 0.10 mol of tranexamic acid and 0.10 mol of mandelic acid are added to acetonitrile for 24 h; under the condition of 50° C., the ultrasonic frequency is 20 kHz, the ultrasonic power is 700 W, the ultrasonic time is 6 h, and the interval time is 10 s every 3 s; and the stirring time is 24 h and the stirring rate is 60 rad/min to obtain a solution of supramolecular tranexamic acid-mandelic acid ionic salt. Under vacuum conditions, the obtained solution of supramolecular tranexamic acid-mandelic acid ionic salt is concentrated and crystallized; then, dried in a vacuum drying oven for 48 h at a drying temperature of 60° C. to obtain supramolecular tranexamic acid-mandelic acid ionic salt.

### Embodiment 3

A supramolecular tranexamic acid-mandelic acid ionic salt, the preparation method of which is as follows: In an inert gas atmosphere, 0.10 mol of tranexamic acid and 0.10 mol of mandelic acid are added to acetonitrile for 24 h; under the condition of 90° C., the ultrasonic frequency is 60 kHz, the ultrasonic power is 6000 W, the ultrasonic time is 12 h, and the interval time is 10 s every 3 s; and the stirring time is 24 h and the stirring rate is 60 rad/min to obtain a solution of supramolecular tranexamic acid-mandelic acid ionic salt. Under vacuum conditions, the obtained solution of supramolecular tranexamic acid-mandelic acid ionic salt is concentrated and crystallized; then, dried in a vacuum drying oven for 48 h at a drying temperature of 60° C. to obtain supramolecular tranexamic acid-mandelic acid ionic salt.

### Embodiment 4

A supramolecular tranexamic acid-mandelic acid ionic salt, which differs from that of Embodiment 1 in that: the volume of tranexamic acid is 6 mol and the volume of mandelic acid is 1 mol.

### Embodiment 5

A supramolecular tranexamic acid-mandelic acid ionic salt crystal, the preparation method of which is as follows.

In an inert gas atmosphere, 0.10 mol of tranexamic acid and 0.10 mol of mandelic acid are added to acetonitrile; under the condition of 75° C., the ultrasonic frequency is 40 kHz, the ultrasonic power is 2000 W, the ultrasonic time is 12 h, and the interval time is 10 s every 3 s; and the stirring time is 24 h and the stirring rate is 60 rad/min to obtain a solution of supramolecular tranexamic acid-mandelic acid ionic salt. Under low-temperature conditions, the obtained solution of supramolecular tranexamic acid-mandelic acid ionic salt is crystallized at a crystallization temperature of 10° C., and supramolecular tranexamic acid mandelic acid crystals are obtained after filtering.

### Comparative Example 1

A supramolecular ionic salt, which differs from that of Embodiment 1 in that: the mandelic acid is replaced with the same volume of citric acid to prepare a supramolecular tranexamic acid-citric acid ionic salt.

### II. MATERIAL PERFORMANCE TESTS

(1) A thermal decomposition behavior of the supramolecular tranexamic acid-mandelic acid ionic salt, tranexamic acid monomer, and mandelic acid monomer is studied using thermogravimetric analysis at a heating rate of 5.0 K/min, and the results are shown in FIG. 2.

As shown in FIG. 2, the supramolecular tranexamic acid-mandelic acid ionic salt begins to undergo the thermal decomposition behavior at around 190° C., indicating that it is stable at room temperature. The thermal decomposition behavior of tranexamic acid and mandelic acid occurs at around 77° C. and 135° C. respectively, that is, the melting point of the supramolecular tranexamic acid-mandelic acid ionic salt is lower than that of tranexamic acid monomer, indicating that the ionic salt is effectively formed.

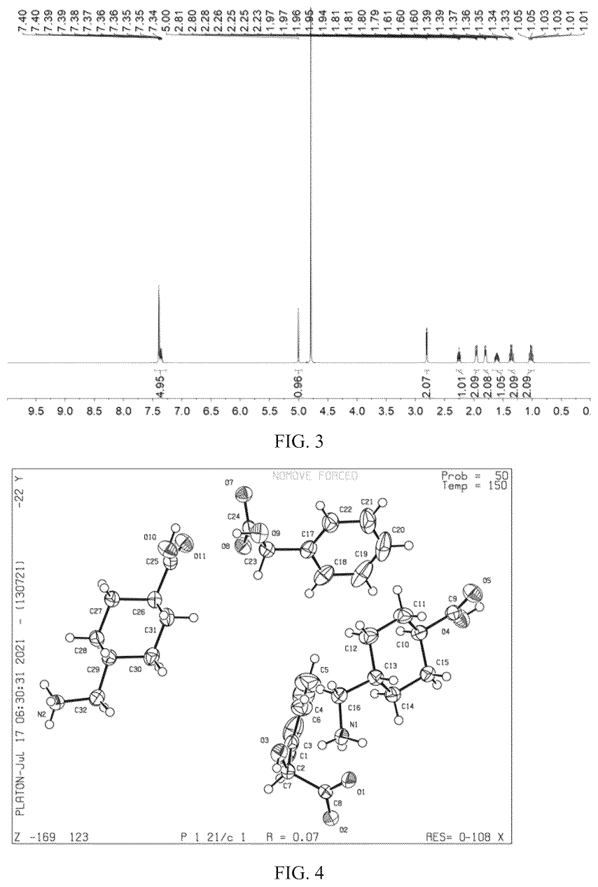

(2) As shown in FIG. 3, the nuclear magnetic hydrogen spectrum data of the supramolecular tranexamic acid-mandelic acid ionic salt prepared in this embodiment are: 1 H NMR (600 MHZ, D2O) δ7.47-7.27 (m, 5 H), 5.00 (s, 1 H), 2.81 (d, J=7.1 Hz, 2 H), 2.25 (tt, J=12.3, 3.5 Hz, 1 H), 1.99-1.92 (m, 2 H), 1.80 (dd, J=9.5, 4.1 Hz, 2 H), 1.69-1.51 (m, 1 H), 1.36 (qd, J=13.0, 3.3 Hz, 2 H), 1.02 (qd, J=12.9, 3.4 Hz, 2 H).

(3) As shown in FIG. 4, supramolecular tranexamic acid mandelic acid crystals obtained in Embodiment 5 are subjected to a single crystal X-ray diffraction test. The specific test parameters are: SuperNova, Dual, Cu at zero, AtlasS2 diffractometer, temperature is 149.99 (10) K, and Olex2 and ShelXL are used for structural analysis.

The single crystal X-ray diffraction test results of the supramolecular tranexamic acid mandelic acid crystals are shown in Table 1.

TABLE 1
Crystal data and structure refinement for supramolecular
tranexamic acid-mandelic acid ionic salt

| Chemical formula | C₁₆H₂₃NO₅ | |
|---|---|---|
| Empirical formula | 309.35 | |
| Formula weight | 149.99 (10) | |
| Crystal system | Orthorhombic | |
| Space group | Pbca | |
| Unit cell parameters | a/Å | 26.126 (2) |
| | b/Å | 6.2881 (5) |
| | c/Å | 21.851 (2) |
| | α/° | 90 |
| | β/° | 114.155 (11) |
| | y/° | 90 |

| | |
|---|---|
| Volume/Å³ | 3275.3 (6) |
| Number of units in the unit cell | 8 |
| Density (calculated value)/g/cm³ | 1.255 |
| Absorption coefficient/mm⁻¹ | 0.769 |
| F(0000) | 1328.0 |
| Crystal size/mm³ | 0.13 × 0.11 × 0.09 |
| Radiation | Cu Ka (λ = 1.54184) |
| Data collection angle range (2θ)/° | 7.416 to 147.964 |
| Index range | −32 ≤ h ≤ 27, −7 ≤ k ≤ 6, −27 ≤ 1 ≤ 26 |
| Reflections collected | 16532 |
| Independent reflections | 6458[Rint = 0.0708, Rsigma = 0.0686] |
| Data/restraints/parameters | 6458/0/403 |
| Goodness-of-fit on F² | 1.062 |
| Final R indexes [I >= 2σ (I)] | R₁ = 0.0719, wR₂ = 0.1808 |
| Final R indexes [all data] | R₁ = 0.0944, wR₂ = 0.2072 |
| Largest diff. peak/hole/e Å⁻³ | 0.33/−0.40 |

The analysis data of atomic coordinates (×10⁴) and equivalent isotropic atomic displacement parameters (Å²×10³) of supramolecular tranexamic acid mandelic acid is shown in Table 2, where U(eq) is defined as one-third of the trace of orthogonal Uᵢⱼ tensor.

TABLE 2
Fractional Atomic Coordinates (×10⁴) and Equivalent Isotropic
Displacement Parameters (Å² × 10³) for supramolecular tranexamic
acid-mandelic acid ionic salt. U_{eq} is defined as ⅓
of the trace of the orthogonalised U_{IJ} tensor.

| Atom | x | | y | | z | | U(eq) | |
|---|---|---|---|---|---|---|---|---|
| O1 | 571.4 | (8) | 10078 | (3) | 2491.7 | (9) | 35.2 | (4) |
| O2 | 768.7 | (8) | 7303 | (3) | 1996.7 | (9) | 34.6 | (4) |
| O3 | 1478.7 | (9) | 12256 | (3) | 2522.5 | (10) | 41.4 | (5) |
| C1 | 1870.6 | (11) | 8988 | (4) | 3078.1 | (14) | 36.0 | (6) |
| C2 | 2084.5 | (13) | 6978 | (5) | 3063.4 | (18) | 48.3 | (7) |
| C3 | 2418.2 | (15) | 5941 | (6) | 3650 | (2) | 66.8 | (11) |
| C4 | 2536.0 | (16) | 6916 | (8) | 4252 | (2) | 78.7 | (13) |
| C5 | 2334.6 | (18) | 8906 | (8) | 4275.7 | (19) | 80.7 | (13) |
| C6 | 2001.0 | (14) | 9959 | (6) | 3689.3 | (16) | 52.8 | (8) |
| C7 | 1466.7 | (11) | 10022 | (4) | 2437.6 | (13) | 32.4 | (5) |
| C8 | 883.2 | (11) | 9077 | (4) | 2287.1 | (12) | 29.3 | (5) |
| O4 | 1035.6 | (10) | 8996 | (3) | 6024.0 | (10) | 44.3 | (5) |
| O5 | 726.8 | (10) | 12098 | (3) | 6218.7 | (10) | 45.9 | (5) |
| N1 | 548.6 | (9) | 14371 | (3) | 2835.8 | (11) | 31.4 | (5) |
| C9 | 912.0 | (11) | 11023 | (4) | 5894.4 | (13) | 35.2 | (6) |
| C10 | 1024.6 | (11) | 11841 | (4) | 5312.7 | (13) | 33.8 | (5) |
| C11 | 1091.9 | (14) | 14238 | (4) | 5332.4 | (14) | 44.2 | (7) |
| C12 | 1213.0 | (14) | 15020 | (4) | 4745.0 | (15) | 44.1 | (7) |
| C13 | 762.7 | (11) | 14310 | (4) | 4067.1 | (13) | 33.7 | (5) |
| C14 | 685.0 | (11) | 11903 | (4) | 4058.6 | (13) | 34.2 | (5) |
| C15 | 558.0 | (12) | 11146 | (4) | 4644.2 | (13) | 35.0 | (6) |
| C16 | 926.8 | (11) | 15135 | (4) | 3516.1 | (13) | 34.7 | (6) |
| O7 | 4416.5 | (8) | 14933 | (3) | 6952.6 | (9) | 35.7 | (4) |
| O8 | 4237.0 | (8) | 12172 | (3) | 6264.5 | (9) | 36.2 | (4) |
| O9 | 3495.8 | (9) | 17042 | (3) | 6068.7 | (11) | 42.8 | (5) |
| C17 | 3124.5 | (11) | 13785 | (4) | 6274.3 | (14) | 36.9 | (6) |
| C18 | 2913.6 | (13) | 11773 | (5) | 6053.4 | (17) | 50.3 | (8) |
| C19 | 2569.4 | (14) | 10780 | (6) | 6319 | (2) | 69.3 | (12) |
| C20 | 2447.0 | (15) | 11807 | (7) | 6800 | (2) | 74.5 | (13) |
| C21 | 2657.6 | (15) | 13798 | (7) | 7018 | (2) | 65.8 | (10) |
| C22 | 2991.2 | (12) | 14794 | (5) | 6750.9 | (16) | 46.6 | (7) |
| C23 | 3523.7 | (11) | 14810 | (4) | 6018.0 | (13) | 32.6 | (5) |
| C24 | 4110.0 | (10) | 13930 | (4) | 6439.0 | (12) | 30.2 | (5) |
| O10 | 3985.5 | (10) | 20880 | (3) | 5041.8 | (10) | 43.7 | (5) |
| O11 | 4272.4 | (9) | 17748 | (3) | 5539.9 | (10) | 44.7 | (5) |
| N2 | 4460.3 | (9) | 15786 | (3) | 2299.4 | (11) | 33.2 | (5) |
| C25 | 4102.3 | (11) | 18841 | (4) | 5038.3 | (13) | 34.2 | (5) |
| C26 | 3997.3 | (11) | 18029 | (4) | 4345.4 | (13) | 32.4 | (5) |
| C27 | 4445.8 | (12) | 18872 | (4) | 4119.3 | (13) | 34.2 | (5) |
| C28 | 4311.2 | (12) | 18145 | (4) | 3401.0 | (13) | 35.1 | (6) |
| C29 | 4271.2 | (11) | 15726 | (4) | 3337.2 | (13) | 32.2 | (5) |
| C30 | 3844.5 | (12) | 14874 | (4) | 3594.5 | (15) | 37.8 | (6) |
| C31 | 3978.2 | (12) | 15613 | (4) | 4309.6 | (14) | 37.9 | (6) |
| C32 | 4096.3 | (11) | 14947 | (4) | 2621.1 | (14) | 35.3 | (6) |

The analysis data of anisotropic atomic displacement parameters for the supramolecular tranexamic acid-mandelic acid ionic salt is shown in Table 3, where the anisotropic atomic displacement factor is expressed as: −2π²[h²a*²U₁₁+2hka*b*U₁₂+ . . . ].

TABLE 3
Anisotropic Displacement Parameters (Å² × 10³) for supramolecular
tranexamic acid-mandelic acid ionic salt. The Anisotropic displacement factor
exponent takes the form: −2π²[h²a*²U₁₁ + 2hka*b*U₁₂ + . . .].

| Atom | U11 | | U22 | | U33 | | U23 | | U13 | | U12 | |
|---|---|---|---|---|---|---|---|---|---|---|---|---|
| O1 | 29.6 | (9) | 39.3 | (9) | 38.5 | (10) | −4.0 | (7) | 15.9 | (8) | −2.7 | (8) |
| O2 | 36.1 | (10) | 34.3 | (9) | 34.2 | (9) | −3.7 | (7) | 15.1 | (8) | −4.8 | (8) |
| O3 | 38.7 | (11) | 38.9 | (10) | 46.6 | (11) | 1.3 | (8) | 17.3 | (9) | −4.4 | (8) |
| C1 | 24.8 | (12) | 42.8 | (14) | 40.7 | (14) | 1.4 | (11) | 13.5 | (11) | −1.9 | (11) |
| C2 | 33.5 | (15) | 46.9 | (16) | 64 | (2) | 0.9 | (14) | 19.4 | (14) | 0.6 | (13) |
| C3 | 36.1 | (18) | 61 | (2) | 95 | (3) | 22 | (2) | 18.2 | (18) | 6.1 | (16) |
| C4 | 38.3 | (19) | 112 | (4) | 69 | (3) | 39 | (2) | 5.0 | (18) | 13 | (2) |
| C5 | 56 | (2) | 128 | (4) | 40.9 | (18) | 0 | (2) | 1.7 | (17) | 12 | (3) |
| C6 | 41.2 | (17) | 66 | (2) | 42.8 | (16) | −6.5 | (14) | 9.3 | (14) | 6.2 | (15) |
| C7 | 30.4 | (13) | 35.8 | (12) | 34.3 | (13) | −4.1 | (10) | 16.5 | (11) | −4.6 | (10) |
| C8 | 29.4 | (12) | 33.5 | (12) | 25.6 | (11) | 0.3 | (9) | 11.8 | (10) | −1.4 | (10) |
| O4 | 61.3 | (14) | 36.3 | (10) | 44.3 | (11) | 3.9 | (8) | 30.9 | (10) | 3.1 | (9) |
| O5 | 59.5 | (14) | 40.3 | (10) | 42.8 | (11) | −0.2 | (8) | 25.8 | (10) | 5.6 | (9) |
| N1 | 28.1 | (11) | 31.4 | (10) | 35.7 | (11) | 2.3 | (8) | 14.0 | (9) | 0.5 | (8) |
| C9 | 35.0 | (14) | 36.1 | (13) | 31.4 | (12) | −1.5 | (10) | 10.3 | (11) | −4.6 | (11) |
| C10 | 33.5 | (13) | 32.7 | (13) | 31.8 | (12) | −0.8 | (10) | 10.0 | (10) | −2.8 | (10) |
| C11 | 57.7 | (19) | 34.1 | (13) | 36.5 | (14) | −4.8 | (11) | 15.1 | (13) | −11.2 | (13) |
| C12 | 50.7 | (18) | 33.9 | (13) | 42.0 | (15) | −1.8 | (11) | 13.3 | (13) | −9.2 | (12) |
| C13 | 32.9 | (13) | 34.6 | (13) | 32.5 | (13) | 1.8 | (10) | 12.4 | (11) | 2.6 | (10) |
| C14 | 35.7 | (14) | 34.0 | (13) | 32.5 | (12) | −1.5 | (10) | 13.5 | (11) | −2.7 | (11) |
| C15 | 35.1 | (14) | 34.9 | (13) | 34.6 | (13) | −1.1 | (10) | 13.9 | (11) | −5.5 | (11) |
| C16 | 32.3 | (13) | 33.7 | (12) | 36.2 | (13) | 1.9 | (10) | 11.9 | (11) | −1.8 | (11) |
| O7 | 30.9 | (9) | 37.1 | (9) | 36.1 | (10) | −1.4 | (7) | 10.6 | (8) | 2.5 | (8) |
| O8 | 38.6 | (10) | 35.8 | (9) | 37.0 | (10) | 0.1 | (7) | 18.2 | (8) | 6.3 | (8) |
| O9 | 39.8 | (11) | 37.6 | (10) | 51.3 | (12) | 8.4 | (8) | 19.1 | (9) | 8.4 | (9) |
| C17 | 24.1 | (12) | 39.8 | (14) | 40.7 | (14) | 7.6 | (11) | 6.9 | (11) | 4.7 | (11) |
| C18 | 31.6 | (15) | 46.1 | (17) | 60.3 | (19) | 4.8 | (13) | 5.7 | (14) | −1.4 | (13) |
| C19 | 31.5 | (17) | 54 | (2) | 101 | (3) | 26 | (2) | 6.2 | (18) | −5.8 | (15) |
| C20 | 32.1 | (17) | 98 | (3) | 96 | (3) | 45 | (3) | 28.5 | (19) | 6.9 | (19) |
| C21 | 38.2 | (18) | 99 | (3) | 70 | (2) | 19 | (2) | 31.7 | (17) | 10.9 | (19) |
| C22 | 31.2 | (14) | 60.5 | (18) | 49.9 | (17) | 2.8 | (14) | 18.6 | (13) | 5.3 | (13) |
| C23 | 30.0 | (13) | 35.2 | (13) | 32.0 | (12) | 1.4 | (10) | 12.0 | (10) | 4.8 | (10) |
| C24 | 27.8 | (12) | 34.9 | (12) | 31.1 | (12) | 3.8 | (10) | 15.2 | (10) | 0.8 | (10) |
| O10 | 60.5 | (13) | 36.5 | (10) | 37.1 | (10) | 0.5 | (8) | 22.8 | (10) | 5.6 | (9) |
| O11 | 54.7 | (13) | 43.0 | (11) | 38.9 | (10) | 7.7 | (8) | 21.8 | (10) | 3.7 | (9) |
| N2 | 30.7 | (11) | 34.0 | (11) | 33.2 | (11) | −2.8 | (8) | 11.2 | (9) | 1.8 | (9) |
| C25 | 31.5 | (13) | 38.3 | (13) | 36.3 | (13) | 2.9 | (11) | 17.4 | (11) | −1.1 | (10) |
| C26 | 32.0 | (13) | 32.2 | (12) | 36.5 | (13) | 1.3 | (10) | 17.5 | (11) | −0.9 | (10) |
| C27 | 35.9 | (14) | 33.9 | (13) | 35.0 | (13) | −0.7 | (10) | 16.7 | (11) | −4.0 | (11) |
| C28 | 39.4 | (14) | 34.7 | (13) | 35.9 | (13) | −2.2 | (10) | 20.2 | (11) | −5.0 | (11) |
| C29 | 28.8 | (13) | 33.4 | (12) | 35.2 | (13) | −1.7 | (10) | 14.0 | (10) | −1.9 | (10) |
| C30 | 37.4 | (15) | 30.7 | (12) | 47.8 | (15) | −2.5 | (11) | 20.0 | (12) | −5.9 | (11) |
| C31 | 40.5 | (15) | 36.1 | (13) | 42.5 | (14) | 3.2 | (11) | 22.6 | (12) | −2.5 | (11) |
| C32 | 30.4 | (13) | 34.6 | (13) | 41.2 | (14) | −3.6 | (10) | 15.0 | (11) | −3.7 | (10) |

The bond length analysis data of various chemical bonds of the supramolecular tranexamic acid-mandelic acid ionic salt is shown in Table 4.

TABLE 4
Bond Lengths for supramolecular tranexamic
acid-mandelic acid ionic salt

| Atom | Atom | Bond length/Å | Atom | Atom | Bond length/Å |
|---|---|---|---|---|---|
| O1 | C8 | 1.248 (3) | O7 | C24 | 1.250 (3) |
| O2 | C8 | 1.258 (3) | O8 | C24 | 1.257 (3) |
| O3 | C7 | 1.416 (3) | O9 | C23 | 1.412 (3) |
| C1 | C2 | 1.387 (4) | C17 | C18 | 1.386 (4) |
| C1 | C6 | 1.379 (4) | C17 | C22 | 1.380 (4) |
| C1 | C7 | 1.512 (4) | C17 | C23 | 1.515 (4) |
| C2 | C3 | 1.383 (5) | C18 | C19 | 1.400 (5) |
| C3 | C4 | 1.369 (6) | C19 | C20 | 1.378 (6) |
| C4 | C5 | 1.367 (6) | C20 | C21 | 1.372 (6) |
| C5 | C6 | 1.388 (5) | C21 | C22 | 1.381 (5) |
| C7 | C8 | 1.541 (3) | C23 | C24 | 1.531 (3) |
| O4 | C9 | 1.317 (3) | O10 | C25 | 1.319 (3) |
| O5 | C9 | 1.214 (3) | O11 | C25 | 1.214 (3) |
| N1 | C16 | 1.488 (3) | N2 | C32 | 1.491 (3) |
| C9 | C10 | 1.507 (4) | C25 | C26 | 1.513 (3) |
| C10 | C11 | 1.516 (3) | C26 | C27 | 1.540 (3) |
| C10 | C15 | 1.534 (3) | C26 | C31 | 1.521 (3) |
| C11 | C12 | 1.524 (4) | C27 | C28 | 1.531 (3) |
| C12 | C13 | 1.533 (4) | C28 | C29 | 1.527 (3) |
| C13 | C14 | 1.526 (3) | C29 | C30 | 1.536 (4) |
| C13 | C16 | 1.525 (4) | C29 | C32 | 1.521 (3) |
| C14 | C15 | 1.524 (3) | C30 | C31 | 1.527 (4) |

The bond angle (°) analysis data of various chemical bonds of the supramolecular tranexamic acid-mandelic acid ionic salt is shown in Table 5.

TABLE 5
Bond Angles for supramolecular tranexamic
acid-mandelic acid ionic salt

| Atom | Atom | Atom | Bond angle/° |
|---|---|---|---|
| C2 | C1 | C7 | 120.4 (3) |
| C6 | C1 | C2 | 118.9 (3) |
| C6 | C1 | C7 | 120.5 (3) |
| C3 | C2 | C1 | 121.0 (3) |
| C4 | C3 | C2 | 119.3 (4) |
| C5 | C4 | C3 | 120.5 (4) |
| C4 | C5 | C6 | 120.6 (4) |
| C1 | C6 | C5 | 119.7 (3) |
| O3 | C7 | C1 | 109.6 (2) |
| O3 | C7 | C8 | 112.2 (2) |
| C1 | C7 | C8 | 106.7 (2) |
| O1 | C8 | O2 | 124.9 (2) |
| O1 | C8 | C7 | 117.6 (2) |
| O2 | C8 | C7 | 117.4 (2) |
| O4 | C9 | C10 | 113.3 (2) |
| O5 | C9 | O4 | 122.3 (2) |
| O5 | C9 | C10 | 124.4 (2) |
| C9 | C10 | C11 | 112.1 (2) |
| C9 | C10 | C15 | 110.6 (2) |
| C11 | C10 | C15 | 110.4 (2) |
| C10 | C11 | C12 | 111.0 (2) |
| C11 | C12 | C13 | 112.2 (2) |
| C14 | C13 | C12 | 110.3 (2) |
| C16 | C13 | C12 | 108.1 (2) |
| C16 | C13 | C14 | 114.0 (2) |
| C15 | C14 | C13 | 111.9 (2) |
| C14 | C15 | C10 | 110.4 (2) |
| N1 | C16 | C13 | 113.1 (2) |
| C18 | C17 | C23 | 119.7 (3) |
| C22 | C17 | C18 | 119.7 (3) |
| C22 | C17 | C23 | 120.5 (3) |
| C17 | C18 | C19 | 119.6 (3) |
| C20 | C19 | C18 | 119.7 (4) |
| C21 | C20 | C19 | 120.5 (3) |
| C20 | C21 | C22 | 120.0 (4) |
| C17 | C22 | C21 | 120.5 (3) |
| O9 | C23 | C17 | 109.2 (2) |
| O9 | C23 | C24 | 112.5 (2) |
| C17 | C23 | C24 | 107.2 (2) |
| O7 | C24 | O8 | 124.4 (2) |
| O7 | C24 | C23 | 118.2 (2) |
| O8 | C24 | C23 | 117.3 (2) |
| O10 | C25 | C26 | 112.7 (2) |
| O11 | C25 | O10 | 123.1 (2) |
| O11 | C25 | C26 | 124.2 (2) |
| C25 | C26 | C27 | 110.8 (2) |
| C25 | C26 | C31 | 112.2 (2) |
| C31 | C26 | C27 | 109.9 (2) |
| C28 | C27 | C26 | 109.9 (2) |
| C29 | C28 | C27 | 111.7 (2) |
| C28 | C29 | C30 | 110.3 (2) |
| C32 | C29 | C28 | 113.3 (2) |
| C32 | C29 | C30 | 107.9 (2) |
| C31 | C30 | C29 | 111.8 (2) |
| C26 | C31 | C30 | 110.2 (2) |
| N2 | C32 | C29 | 113.0 (2) |

The hydrogen bond analysis data of the supramolecular tranexamic acid-mandelic acid ionic salt is shown in Table 6.

TABLE 6
Hydrogen bonds of supramolecular tranexamic
acid-mandelic acid ionic salt

| | | | d(D − | d(H − | d(D − | D − |
|---|---|---|---|---|---|---|
| D | H | A | H)/Å | A)/Å | A)/Å | H − A/° |
| O3 | H3 | O51 | 0.82 | 1.98 | 2.750 (3) | 156.0 |
| O4 | H4 | O22 | 0.82 | 1.81 | 2.624 (3) | 175.8 |
| N1 | H1A | O23 | 0.89 | 1.94 | 2.820 (3) | 168.0 |
| N1 | H1B | O14 | 0.89 | 1.87 | 2.749 (3) | 168.1 |
| N1 | H1C | O1 | 0.89 | 1.94 | 2.810 (3) | 163.6 |
| O9 | H9 | O11 | 0.82 | 1.98 | 2.751 (3) | 157.1 |
| O10 | H10A | O83 | 0.82 | 1.79 | 2.606 (3) | 178.7 |
| N2 | H2A | O81 | 0.89 | 1.92 | 2.799 (3) | 166.9 |
| N2 | H2B | O75 | 0.89 | 1.89 | 2.750 (3) | 162.7 |
| N2 | H2C | O76 | 0.89 | 1.93 | 2.786 (3) | 160.3 |

The torsion angle (°) analysis data of various chemical bonds of the supramolecular

tranexamic acid-mandelic acid ionic salt is shown in Table 7.

TABLE 7
Torsion Angles for supramolecular tranexamic acid-mandelic acid ionic salt

| A | B | C | D | Angle/° | | A | B | C | D | Angle/° | |
|---|---|---|---|---|---|---|---|---|---|---|---|
| O3 | C7 | C8 | O1 | −25.7 | (3) | O9 | C23 | C24 | O7 | 28.1 | (3) |
| O3 | C7 | C8 | O2 | 157.7 | (2) | O9 | C23 | C24 | O8 | −156.1 | (2) |
| C1 | C2 | C3 | C4 | 0.3 | (5) | C17 | C18 | C19 | C20 | −0.5 | (5) |
| C1 | C7 | C8 | O1 | 94.3 | (3) | C17 | C23 | C24 | O7 | −91.9 | (3) |
| C1 | C7 | C8 | O2 | −82.3 | (3) | C17 | C23 | C24 | O8 | 83.9 | (3) |
| C2 | C1 | C6 | C5 | −0.9 | (5) | C18 | C17 | C22 | C21 | 1.1 | (5) |
| C2 | C1 | C7 | O3 | −157.3 | (2) | C18 | C17 | C23 | O9 | 155.5 | (2) |
| C2 | C1 | C7 | C8 | 81.1 | (3) | C18 | C17 | C23 | C24 | −82.4 | (3) |
| C2 | C3 | C4 | C5 | −1.1 | (6) | C18 | C19 | C20 | C21 | 0.3 | (6) |
| C3 | C4 | C5 | C6 | 0.9 | (7) | C19 | C20 | C21 | C22 | 0.5 | (6) |
| C4 | C5 | C6 | C1 | 0.1 | (7) | C20 | C21 | C22 | C17 | −1.3 | (5) |
| C6 | C1 | C2 | C3 | 0.7 | (5) | C22 | C17 | C18 | C19 | −0.3 | (4) |
| C6 | C1 | C7 | O3 | 27.3 | (4) | C22 | C17 | C23 | O9 | −27.8 | (3) |
| C6 | C1 | C7 | C8 | −94.3 | (3) | C22 | C17 | C23 | C24 | 94.3 | (3) |
| C7 | C1 | C2 | C3 | −174.8 | (3) | C23 | C17 | C18 | C19 | 176.5 | (3) |
| C7 | C1 | C6 | C5 | 174.5 | (3) | C23 | C17 | C22 | C21 | −175.6 | (3) |
| O4 | C9 | C10 | C11 | −158.5 | (2) | O10 | C25 | C26 | C27 | 72.3 | (3) |
| O4 | C9 | C10 | C15 | 77.8 | (3) | O10 | C25 | C26 | C31 | −164.4 | (2) |
| O5 | C9 | C10 | C11 | 21.4 | (4) | O11 | C25 | C26 | C27 | −108.1 | (3) |
| O5 | C9 | C10 | C15 | −102.3 | (3) | O11 | C25 | C26 | C31 | 15.2 | (4) |
| C9 | C10 | C11 | C12 | 179.2 | (2) | C25 | C26 | C27 | C28 | −176.5 | (2) |
| C9 | C10 | C15 | C14 | −177.8 | (2) | C25 | C26 | C31 | C30 | 177.4 | (2) |
| C10 | C11 | C12 | C13 | 55.7 | (3) | C26 | C27 | C28 | C29 | −57.3 | (3) |
| C11 | C10 | C15 | C14 | 57.6 | (3) | C27 | C26 | C31 | C30 | −58.9 | (3) |
| C11 | C12 | C13 | C14 | −53.8 | (3) | C27 | C28 | C29 | C30 | 54.8 | (3) |
| C11 | C12 | C13 | C16 | −179.0 | (2) | C27 | C28 | C29 | C32 | 175.8 | (2) |
| C12 | C13 | C14 | C15 | 54.5 | (3) | C28 | C29 | C30 | C31 | −54.6 | (3) |
| C12 | C13 | C16 | N1 | 174.5 | (2) | C28 | C29 | C32 | N2 | 54.0 | (3) |
| C13 | C14 | C15 | C10 | −56.9 | (3) | C29 | C30 | C31 | C26 | 57.2 | (3) |
| C14 | C13 | C16 | N1 | 51.5 | (3) | C30 | C29 | C32 | N2 | 176.4 | (2) |
| C15 | C10 | C11 | C12 | −57.0 | (3) | C31 | C26 | C27 | C28 | 59.0 | (3) |
| C16 | C13 | C14 | C15 | 176.4 | (2) | C32 | C29 | C30 | C31 | −178.8 | (2) |

The analysis data of the hydrogen atomic coordinates (Å×10⁴) and various isotropic atomic displacement parameters (Å×10³) of the supramolecular tranexamic acid-mandelic acid ionic salt is shown in Table 8.

TABLE 8
Hydrogen Atom Coordinates (Å × 10⁴) and
Isotropic Displacement Parameters (Å² ×
10³) for supramolecular tranexamic acid-mandelic acid ionic salt

| Atom | x | y | z | U(eq) |
|---|---|---|---|---|
| H3 | 1264.62 | 12815.63 | 2172.1 | 62 |
| H2 | 2002.18 | 6318.21 | 2652.98 | 58 |
| H3A | 2561.04 | 4597.21 | 3634.57 | 80 |
| H4A | 2754.66 | 6218.05 | 4649.15 | 94 |
| H5 | 2421.83 | 9560.13 | 4688.01 | 97 |
| H6 | 1865.91 | 11314.62 | 3708.57 | 63 |
| H7 | 1576.2 | 9669.66 | 2070.97 | 39 |
| H4 | 964.7 | 8629.64 | 6340.99 | 66 |
| H1A | 603.26 | 15147.11 | 2527.17 | 38 |
| H1B | 193.37 | 14497.23 | 2782.12 | 38 |
| H1C | 622.95 | 13013.12 | 2791.38 | 38 |
| H10 | 1376.98 | 11207.92 | 5342.8 | 41 |
| H11A | 1397.25 | 14648.65 | 5750.32 | 53 |
| H11B | 751.19 | 14901.27 | 5315.54 | 53 |
| H12A | 1574.78 | 14479.57 | 4791.29 | 53 |
| H12B | 1233.19 | 16560.83 | 4756.57 | 53 |
| H13 | 407.26 | 14979.71 | 4009.8 | 40 |
| H14A | 379.49 | 11493.14 | 3640.72 | 41 |
| H14B | 1023.55 | 11212.44 | 4078.91 | 41 |
| H15A | 526.53 | 9607.89 | 4632.5 | 42 |
| H15B | 202.68 | 11735.2 | 4604.54 | 42 |
| H16A | 921.63 | 16677.22 | 3519.59 | 42 |
| H16B | 1307.22 | 14686.65 | 3611.97 | 42 |
| H9 | 3708.13 | 17603.45 | 5925.47 | 64 |
| H18 | 3000.16 | 11085.51 | 5730.89 | 60 |
| H19 | 2424.01 | 9433.63 | 6170.85 | 83 |
| H20 | 2220.13 | 11145.63 | 6978.18 | 89 |
| H21 | 2575.73 | 14476.59 | 7345.13 | 79 |
| H22 | 3127.13 | 16154.8 | 6893.47 | 56 |
| H23 | 3413.89 | 14416.74 | 5547.16 | 39 |
| H10A | 4064.67 | 21267.76 | 5428.27 | 66 |
| H2A | 4421.25 | 14977.13 | 1948.76 | 40 |
| H2B | 4816.79 | 15765.72 | 2594.73 | 40 |
| H2C | 4360.06 | 17114.69 | 2162.85 | 40 |
| H26 | 3631.96 | 18569.13 | 4032.12 | 39 |
| H27A | 4455.22 | 20412.9 | 4140 | 41 |
| H27B | 4812.08 | 18342.88 | 4417.87 | 41 |
| H28A | 3958.13 | 18769 | 3099.9 | 42 |
| H28B | 4601.61 | 18648.43 | 3267.85 | 42 |
| H29 | 4639.8 | 15123.11 | 3614.01 | 39 |
| H30A | 3471.96 | 15357.76 | 3300.43 | 45 |
| H30B | 3845 | 13331.79 | 3582.48 | 45 |
| H31A | 3693.58 | 15090.09 | 4449.68 | 45 |
| H31B | 4337.37 | 15036.93 | 4611.99 | 45 |
| H32A | 4109.12 | 13405.25 | 2621.57 | 42 |
| H32B | 3711.75 | 15375.87 | 2356.58 | 42 |

### III. APPLICATION TEST EXAMPLES

### Test Example 1

A supramolecular tranexamic acid-mandelic acid ionic salt prepared in Embodiment 1 is prepared into a solution of 10 wt % supramolecular tranexamic acid mandelic acid, and an equivalent molar volume of 4.92 wt % mandelic acid solution is prepared for comparison. The above solutions are tested for transdermal effect, and the specific test method is as follows: I. The back skin of GF Kunming mice is carefully stripped of the subcutaneous fat layer and connective tissue, washed with physiological saline, and then placed in the saline for later use. II. The transdermal experiment is conducted using the Franz cell method, with the exposed area of mouse skin in the Franz diffusion apparatus being 1.13 cm² and the receptor chamber volume being 15 mL. III. A volume of 1.0 mL of the solution of supramolecular tranexamic acid mandelic acid and an equivalent molar volume of the mandelic acid solution, prepared as described above, is separately placed in the diffusion cell on the exposed surface of the skin, and 15 mL of physiological saline is added to the receptor chamber. The setup is placed in a constant temperature water bath at 32±0.5° C., with a stirring speed of 350 rad/min. IV. Subcutaneous permeation amount test: 1 mL of receptor solution is taken at different time points, with three parallel samples for each time point. Immediately after sampling, 1 mL of receptor solution is replenished into the receptor chamber. The solution is filtered through a 0.22 μm microporous filter membrane and analyzed by HPLC, and the cumulative permeation amount of mandelic acid is calculated using the following Formula (1):

$$Q = Cn \times V + \sum Ci \times {V}_{0} ( i = 1 … n - 1 ) . \qquad Formula ( 2 )$$

Q: cumulative permeation amount, μg; V: volume of the receptor solution in the receptor chamber, 15 mL; Vo: volume taken for each sampling, 1.0 mL; Ci: medicine concentration in the receptor solution during the first to the (n-1)th sampling; and Cn: concentration of the sample measured at the nth sampling point.

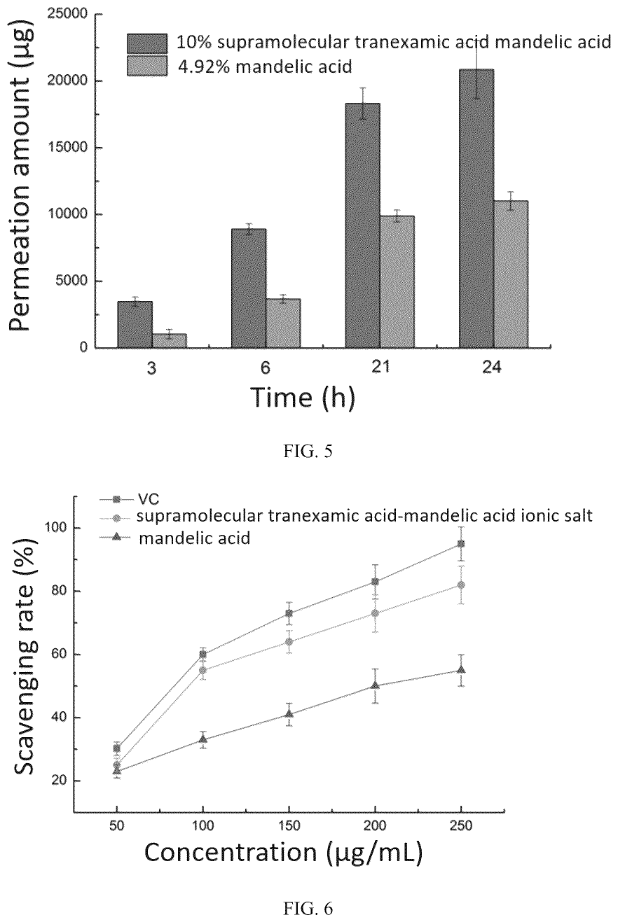

FIG. 5 is a statistical diagram of the permeation effect. It can be seen from FIG. 5 that the permeation amount of mandelic acid in solution of 10% supramolecular tranexamic acid mandelic acid is higher than in the equivalent molar volume of the mandelic acid solution in each time period, and the cumulative permeation amount of mandelic acid at the 24th hour is 1.89 times that of an equivalent molar volume of the mandelic acid solution.

The supramolecular ionic salts of Embodiments 1 to 4 and Comparative Example 1 are respectively prepared into solutions of 10 wt % supramolecular ionic salt, which are named as solution 1 of supramolecular tranexamic acid mandelic acid, solution 2 of supramolecular tranexamic acid mandelic acid, solution 3 of supramolecular tranexamic acid mandelic acid, solution 4 of supramolecular tranexamic acid mandelic acid, and solution 1 of supramolecular tranexamic acid citric acid in sequence. The transdermal effect test is carried out according to the above method, and the test results are shown in Table 9.

TABLE 9
Statistics of permeation effect

| | Permeation |
|---|---|
| Substance | amount (μg) |
| Solution 1 of supramolecular tranexamic acid mandelic acid | 20700 |
| Solution 2 of supramolecular tranexamic acid mandelic acid | 13500 |
| Solution 3 of supramolecular tranexamic acid mandelic acid | 15500 |
| Solution 4 of supramolecular tranexamic acid mandelic acid | 17000 |
| Solution 1 of supramolecular tranexamic acid citric acid | 11300 |

Combined with the comparison between Embodiments 1 to 4 and Comparative Example 1, as well as the comparison between Embodiments 1 and 4, it can be seen that when the mole ratio of tranexamic acid to mandelic acid is in the range of 1:5 to 5:1, the prepared supramolecular tranexamic acid mandelic acid has a better permeation effect, indicating that a specific ratio of tranexamic acid and mandelic acid can play a role in improving the permeation effect, and this effect is stronger than ionic salts made of other ligands, such as supramolecular tranexamic acid citric ionic salt.

### Test Example 2

According to T/SHRH027-2019 In Vitro Test—Inhibition of Melanin Synthesis in B16 Cells Test and T-SHRH015-2018 Cosmetics—Tyrosinase Activity Inhibition Test Method, the maximum safe dose of supramolecular tranexamic acid-mandelic acid ionic salt in Embodiment 1 and supramolecular tranexamic acid citric ionic salt in Comparative Example 1 on melanoma cells is determined based on the cytotoxicity test by melanoma cells (B16). And the cellular melanin content, tyrosinase activity, and tyrosinase inhibition rate are tested within a safe concentration.

The cytotoxicity test results are shown in Tables 10 and 11 below.

TABLE 10
Cytotoxicity test results of supramolecular tranexamic acid-mandelic acid ionic salt

| Cell | | PC | | | | | | | | |
|---|---|---|---|---|---|---|---|---|---|---|
| viability | Control | 10% | Concentration gradient (%, m/V) | | | | | | | |
| (%) | / | DMSO | 0.078 | 0.039 | 0.020 | 0.010 | 0.005 | 0.002 | 0.0012 | 0.0006 |
| Mean | 100.00 | 40.26 | 76.64 | 76.55 | 74.32 | 70.23 | 72.19 | 80.32 | 81.31 | 81.59 |
| SD | 7.57 | 2.58 | 2.74 | 1.32 | 3.25 | 2.01 | 4.92 | 3.19 | 3.17 | 4.93 |

TABLE 11
Cytotoxicity test results of supramolecular tranexamic acid-citric acid ionic salt

| Cell | | PC | | | | | | | | |
|---|---|---|---|---|---|---|---|---|---|---|
| viability | Control | 10% | Concentration gradient (%, m/V) | | | | | | | |
| (%) | / | DMSO | 0.078 | 0.039 | 0.020 | 0.010 | 0.005 | 0.002 | 0.0012 | 0.0006 |
| Mean | 100.00 | 39.46 | 75.85 | 75.13 | 72.19 | 70.74 | 71.95 | 80.01 | 80.58 | 81.32 |
| SD | 7.19 | 3.15 | 2.88 | 1.43 | 2.76 | 2.95 | 3.64 | 2.87 | 3.46 | 4.55 |

Based on the cytotoxicity results, both samples did not show melanoma cytotoxicity in the concentration range of 0.002% (m/V).

The results of the melanin synthesis inhibition assay in cells are as follows:

TABLE 12
Analysis of the inhibition rate of melanin synthesis in cells

| | Concentration | Inhibition rate of cell | | |
|---|---|---|---|---|
| Sample name | (%, m/V) | melanin content (%) | SD | p-value |
| Control | BC | 0.00 | 3.05 | / |
| | PC | 33.28 | 1.53 | 0.010** |
| Supramolecular | 0.0006 | 19.28 | 1.12 | 0.021* |
| tranexamic | | | | |
| acid-mandelic | 0.0012 | 23.51 | 2.07 | 0.006** |
| acid ionic salt | 0.0020 | 31.37 | 2.48 | 0.004** |
| Supramolecular | 0.0006 | 15.32 | 1.62 | 0.031* |
| tranexamic | | | | |
| acid-citric acid | 0.0012 | 18.19 | 2.16 | 0.020* |
| ionic salt | 0.0020 | 22.51 | 3.42 | 0.007** |

Note:
When statistical analysis is performed using the t-test, the significance of the PC group and sample group compared with the BC group is denoted by * as follows: *for p-value < 0.05, and **for p-value < 0.01.

The results of cellular tyrosinase activity inhibition are as follows.

TABLE 13
Results of the cellular tyrosinase activity inhibition assay tyrosinase

| | Concentration | Cellular tyrosinase | | |
|---|---|---|---|---|
| Sample name | (%, m/V) | activity inhibition (%) | SD | p-value |
| Control | BC | 0.00 | 2.04 | / |
| | PC | 51.21 | 2.47 | 0.000** |
| Supramolecular | 0.0006% | 4.91 | 2.16 | 0.025* |
| tranexamic | | | | |
| acid-mandelic | 0.0012% | 19.94 | 5.61 | 0.019* |
| acid ionic salt | 0.0020% | 16.25 | 1.65 | 0.009** |
| Supramolecular | 0.0006% | 2.17 | 1.01 | 0.04* |
| tranexamic | | | | |
| acid-citric acid | 0.0012% | 10.05 | 1.58 | 0.02* |
| ionic salt | 0.0020% | 11.26 | 3.26 | 0.025* |

The results of the in vitro tyrosinase inhibition assay are as follows:

TABLE 14
Analysis of in vitro tyrosinase inhibition assay results tyrosinase

| | Concentration | Cellular tyrosinase | | |
|---|---|---|---|---|
| Sample name | (%, m/V) | activity inhibition (%) | SD | p-value |
| Control | BC | 0.00 | 16.93 | / |
| | PC | 81.27 | 5.14 | 0.007** |
| Supramolecular | 0.625 | 15.29 | 3.64 | 0.185 |
| tranexamic | 1.25 | 83.45 | 1.57 | 0.007** |
| acid-mandelic | 2.50 | 84.42 | 4.32 | 0.005** |
| acid ionic salt | 5 | 99.75 | 3.29 | 0.006** |
| | 10 | 99.61 | 0.59 | 0.008** |
| Supramolecular | 0.625 | 5.36 | 0.31 | 0.02* |
| tranexamic | 1.25 | 60.49 | 3.25 | 0.009** |
| acid-citric acid | 2.50 | 65.38 | 6.25 | 0.009** |
| ionic salt | 5 | 67.57 | 4.21 | 0.008** |
| | 10 | 69.99 | 3.95 | 0.007** |

In Tables 12 to 14, the BC group is a blank control group and the PC group is a positive control group.

The results of the melanin synthesis inhibition assay in cells are shown in Table 12. After the cells are treated with 0.0006%, 0.0012%, and 0.002% (m/V) of supramolecular tranexamic acid-mandelic acid ionic salt and supramolecular tranexamic acid citric ionic salt, the inhibition rate of melanin synthesis increases with the increase of sample concentration, and there is a statistically significant difference compared with the blank control (p<0.05), indicating that it had an inhibitory effect on the cell melanin synthesis ability. And the inhibition rate of supramolecular tranexamic acid-mandelic acid ionic salt on melanin synthesis is higher than that of supramolecular tranexamic acid citric ionic salt.

The results of the cellular tyrosinase activity inhibition assay are shown in Table 13. After the cells are treated with 0.0006%, 0.0012%, and 0.002% (m/V) supramolecular tranexamic acid-mandelic acid ionic salt and supramolecular tranexamic acid-citric acid ionic salt, the inhibition rate of cellular tyrosinase activity increases with the increase of sample concentration, and there is a statistically significant difference compared with the blank control (p<0.05), indicating that it had an inhibitory effect on cellular tyrosinase activity. And the inhibition rate of supramolecular tranexamic acid-mandelic acid ionic salt on cellular tyrosinase activity is higher than that of supramolecular tranexamic acid-citric acid ionic salt.

As shown in Table 14, the inhibition rate of supramolecular tranexamic acid-mandelic acid ionic salt on tyrosinase at concentrations of 1.25 to 10% (m/V) is above 80%, which is statistically different from that of the blank control group (p<0.01), indicating that it has an inhibitory effect on tyrosinase activity; the inhibition rate of supramolecular tranexamic acid citric ionic salt on tyrosinase at concentrations of 1.25 to 10% (m/V) is above 60%, indicating that the inhibitory effect of supramolecular tranexamic acid-mandelic acid ionic salt on tyrosinase activity is higher than that of supramolecular tranexamic acid-citric acid ionic salt.

### Test Example 3

The anti-inflammatory effects of the supramolecular tranexamic acid-mandelic acid ionic salt prepared in Embodiment 1 are tested as follows.

TPA-induced mouse ear edema model and administration method. Male KM mice of SPF grade weighing 20 g to 23 g are acclimatized at a temperature of 25° C. and a relative humidity of 40% to 70% for 24 h (12 h each under alternating light and dark conditions), and given sufficient food and water to allow them to eat freely. The mice are randomly divided into 6 groups: blank group (BC), negative control group (NC), positive control group (PC), and 2 treatment groups. The supramolecular tranexamic acid-mandelic acid ionic salt (0.8 mg/ear) and mandelic acid (0.39 mg/ear) are evenly applied inside and outside the right ear of the mice in the treatment groups, and 2.0 μg/ear of phorbol ester (TPA) is evenly applied inside and outside the right ear of the mice 15 min later to cause inflammation. After 6 h, the mice are immediately killed by breaking their necks. The ear tissues are ground in liquid nitrogen, then ground tissues are transferred to a 4 mL EP tube, and 1 mL of T-PER tissue protein extraction reagent is added. The solution is mixed thoroughly for about 30 min, and then subjected to intermittent sonication for 10 s at 20 Hz, followed by 2 min of ultrasonic treatment to fully lyse the ear tissues. The mixture is centrifuged at 4° C. and 13000 r/min for 20 min. Supernatants are collected to determine the levels of the inflammatory factors IL-1α, TNF-α, and MIP-2 in ear tissues using the ELISA method.

The results of the effects of supramolecular tranexamic acid-mandelic acid ionic salt and mandelic acid monomer on the inflammatory factors IL-1α, TNF-α, and MIP-2 in mouse ear tissues are as follows:

TABLE 15
Summary of IL-1α test results

| | | | | | Concen- |
|---|---|---|---|---|---|
| | | | | Concentration | tration |
| Group | R1 | R2 | R3 | (μg/mL) | SD |
| BC | 12.05 | 12.64 | 11.94 | 12.21 | 0.38 |
| NC | 38.25 | 40.31 | 37.64 | 38.73 | 1.40 |
| PC | 19.64 | 21.37 | 20.82 | 20.61 | 0.88 |
| Supramolecular | 22.34 | 23.16 | 21.87 | 22.46 | 0.65 |
| tranexamic | | | | | |
| acid-mandelic acid | | | | | |
| ionic salt | | | | | |
| Mandelic acid | 28.59 | 27.64 | 29.31 | 28.51 | 0.84 |

TABLE 16
Summary of TNF-α test results

| | | | | Concen- | Concen- |
|---|---|---|---|---|---|
| | | | | tration | tration |
| Group | R1 | R2 | R3 | (μg/mL) | SD |
| BC | 16.25 | 16.94 | 17.21 | 16.80 | 0.50 |
| NC | 109.64 | 108.31 | 107.26 | 108.40 | 1.19 |
| PC | 52.21 | 55.34 | 53.46 | 53.67 | 1.58 |
| Supramolecular | 70.34 | 72.51 | 71.35 | 71.40 | 1.09 |
| tranexamic | | | | | |
| acid-mandelic acid | | | | | |
| ionic salt | | | | | |
| Mandelic acid | 85.34 | 84.33 | 88.27 | 85.98 | 2.05 |

TABLE 17
Summary of MIP-2 test results

| | | | | | Concen- |
|---|---|---|---|---|---|
| | | | | Concentration | tration |
| Group | R1 | R2 | R3 | (μg/mL) | SD |
| BC | 22.61 | 20.16 | 23.64 | 22.14 | 1.79 |
| NC | 68.34 | 69.54 | 64.38 | 67.42 | 2.70 |
| PC | 33.22 | 35.12 | 34.17 | 34.17 | 0.95 |
| Supramolecular | 40.22 | 43.15 | 41.25 | 41.54 | 1.49 |
| tranexamic | | | | | |
| acid-mandelic acid | | | | | |
| ionic salt | | | | | |
| Mandelic acid | 51.32 | 50.16 | 50.98 | 50.82 | 0.60 |

The BC group is a blank control group, the NC group is a negative control group, and the PC group is a positive control group. As can be seen from Tables 15 to 17, the levels of IL-1α, TNF-α, and MIP-2 in the negative control group significantly increase compared with the blank group; compared with the negative control group, the levels of IL-1α, TNF-α, and MIP-2 in the positive control group significantly decrease; this indicates that the experiment is effective. Compared with the negative control group, the levels of IL-1α, TNF-α, and MIP-2 of the supramolecular tranexamic acid-mandelic acid ionic salt group and the mandelic acid monomer group of the present application decrease, while the levels of IL-1α, TNF-α, and MIP-2 of the supramolecular tranexamic acid-mandelic acid ionic salt group decrease more significantly. Under the same mandelic acid content, the effect of the supramolecular tranexamic acid-mandelic acid ionic salt on reducing IL-1α, TNF-α, and MIP-2 is 1.59 times, 1.65 times, and 1.56 times that of the mandelic acid monomer, respectively. This indicates that both the supramolecular tranexamic acid-mandelic acid ionic salt and the mandelic acid monomer have anti-inflammatory effects, with the supramolecular tranexamic acid-mandelic acid ionic salt showing a more significant anti-inflammatory effect on the inflammatory factors IL-1α, TNF-α, and MIP-2.

### Test Example 4

The supramolecular tranexamic acid-mandelic acid ionic salt and mandelic acid monomer obtained from Embodiment 1 are prepared into solutions at concentrations of 50 μg/mL, 100 μg/mL, 150 μg/mL, 200 μg/mL, and 250 μg/mL. The DPPH⋅ radical scavenging rate and ABTS⁺⋅ scavenging rate are measured, and the test methods are as follows.

DPPH⋅ radical scavenging rate determination: For each sample solution at different mass concentrations, 0.2 mL is taken and mixed with 0.8 mL of a 60 μmol/L DPPH⋅ solution (prepared with absolute ethanol). The mixture is then reacted for 30 min in the dark, and the absorbance is measured at a wavelength of 517 nm as A₁. The absorbance using 70% ethanol solution instead of the sample is measured as A₀, and the absorbance using absolute ethanol instead of the DPPH⋅ solution is measured as A₂. VC is used as a positive control during the test. The calculation of the DPPH⋅ radical scavenging rate is shown in Formula (2):

$$scavenging rate / \% = \frac{{A}_{0} - {A}_{1} + {A}_{2}}{{A}_{0}} . \qquad Formula ( 2 )$$

ABTS⁺⋅ scavenging rate determination: ABTS solution (7 mmol/L) and potassium persulfate solution (2.45 mmol/L) are mixed in a 1:1 (v/v) ratio and shaken to prepare the ABTS stock solution, which is allowed to sit at room temperature for 12 h to 16 h (protected from light). Then, the ABTS stock solution is diluted with a phosphate buffer solution of 10 mmol/L and pH 7.4, and the absorbance A₀ of the diluted solution is measured at a wavelength of 734 nm, aiming to reach 0.70±0.002. For each sample solution at different mass concentrations, 50 μL is taken and mixed with 750 μL of the ABTS assay solution. The mixture is thoroughly mixed and allowed to react for 6 min. The absorbance is measured at a wavelength of 734 nm as A₁, with an absolute ethanol solution used in place of the ABTS solution to measure the absorbance as A₂. VC is used as a positive control during the test, and the calculation of the ABTS⁺⋅ scavenging rate follows Formula (2).

The results of DPPH. radical scavenging rate are shown in FIG. 6. The supramolecular tranexamic acid-mandelic acid ionic salt, mandelic acid, and VC all have the ability to scavenge DPPH⋅ radicals, with VC exhibiting the best scavenging effect, followed by the supramolecular tranexamic acid-mandelic acid ionic salt, and mandelic acid showing the weakest effect. Moreover, the higher the mass concentration of the supramolecular tranexamic acid-mandelic acid ionic salt, the better the scavenging effect on DPPH⋅ radicals. This is because the sample can be paired with a single electron present in the DPPH⋅ radical, leading to its reduction and thereby weakening the color of the purple ethanol solution. The absorbance is measured at a wavelength of 517 nm, and the greater the change in absorbance, the stronger the scavenging ability. Therefore, at the same concentration, the supramolecular tranexamic acid-mandelic acid ionic salt exhibits a better scavenging effect on DPPH⋅ than the mandelic acid monomer.

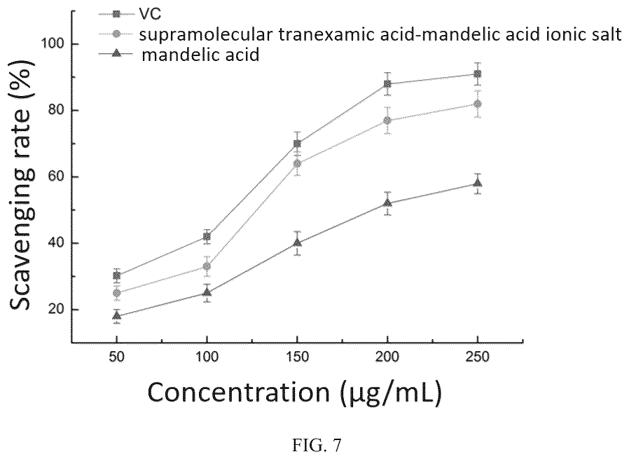

The results of the ABTS⁺⋅ scavenging rate are shown in FIG. 7. The supramolecular tranexamic acid-mandelic acid ionic salt, mandelic acid, and VC all have significant effects in scavenging ABTS⁺, with VC exhibiting the best scavenging effect, followed by the supramolecular tranexamic acid-mandelic acid ionic salt, and mandelic acid showing the weakest effect. ABTS reacts with the oxidant to form the ABTS⁺⋅ radical (blue-green), and the sample reduces ABTS⁺⋅ to colorless ABTS, and its absorbance is measured at a wavelength of 734 nm or 405 nm. The greater the change in absorbance, the stronger the scavenging ability. Therefore, at the same concentration, the supramolecular tranexamic acid-mandelic acid ionic salt exhibits a better scavenging effect on ABTS⁺⋅ than the mandelic acid monomer.

### Test Example 5

The tranexamic acid mandelic acid ionic salt obtained in Embodiment 1 is prepared into a 2% aqueous solution, and an equivalent molar volume of mandelic acid solution is prepared to conduct cosmetic eye irritation/corrosion chick embryo chorioallantoic membrane assay comparison. The test method is as follows.

Preparation of CAM: The viability of 9-day-old chick embryos is checked using fertilized eggs. The shell of the air cell is removed using dental serrated forceps to expose the white egg membrane, with care taken to avoid damaging the integrity of the membrane. A drop of 0.9% sodium chloride solution is added with a pipette to moisten the membrane. The inner membrane is carefully removed with forceps, ensuring that the vascular membrane is not damaged. At this point, the structure of the vascular system is observed again, and a judgment is made regarding its integrity and suitability for the test.

Pre-test: Two chick embryos are taken to assess the reactivity of this batch, with the duration of action limited to within 5 min.

Endpoint evaluation method: 0.3 mL or 0.3 g of the test substance is applied to the CAM, ensuring that at least 50% of the CAM surface is covered by the test substance. After 3 min of application, the test substance on the CAM is gently rinsed with saline, and approximately 30 s after rinsing, the extent of changes in each type of toxicity effect is observed.

Reaction evaluation method: 0.3 mL or 0.3 g of the test substance is applied to the CAM, ensuring that at least 50% of the CAM surface is covered by the test substance. The reaction of the CAM is observed, and the time at which each type of toxicity effect appears within 5 min of application is recorded.

Each sample is placed with 6 chick embryos, and 1 chick embryo is set up for the negative control and 1 for the positive control.

The irritation score (IS) is a test conducted using the reaction time method, with the irritation score (IS) calculated using Formula (3), and the results are retained to two decimal places:

$$IS = \frac{) 1 - sec H ) \times 5}{300} + \frac{( 301 - sec L ) \times 7}{300} + \frac{( 301 - sec C ) \times 9}{300} . \qquad Formula ( 3 )$$

secH (bleeding time) refers to the average time observed for bleeding to begin on the CAM membrane, measured in seconds(s). secL (vascular lysis time) refers to the average time observed for vascular lysis to begin on the CAM membrane, measured in seconds(s). secC (clotting time) refers to the average time observed for clotting to begin on the CAM membrane, measured in seconds(s).

As shown in Table 18, the eye irritation of the test substance is classified based on the calculated IS values.

TABLE 18
Irritation classification

| Irritation score | Irritation classification |
|---|---|
| IS < 1 | No irritation |
| 1 ≤ IS < 5 | Mild irritation |
| 5 ≤ IS < 10 | Moderate irritation |
| IS ≥ 10 | Severe irritation/corrosive |

The test results are as follows.

TABLE 19
Irritation scores and results

| | | | Vas- | | | |
|---|---|---|---|---|---|---|
| | | Bleeding | cular | Coagu- | | |
| Reaction time | | severity | lysis | lation | | |
| method | | score | score | score | IS | Test result |
| Supramolecular | 1 | 301 | 270 | 301 | 0.72 | IS = 0.68 |
| tranexamic | 2 | 301 | 301 | 301 | 0.00 | Result |
| acid-mandelic | 3 | 301 | 301 | 301 | 0.00 | evaluation: |
| acid | 4 | 301 | 301 | 301 | 0.00 | no |
| ionic salt | 5 | 301 | 301 | 301 | 0.00 | irritation |
| | 6 | 301 | 210 | 260 | 3.53 | |
| Mandelic | 1 | 301 | 260 | 301 | 0.96 | IS = 1.03 |
| acid | 2 | 301 | 280 | 301 | 0.49 | Result |
| | 3 | 301 | 280 | 290 | 0.82 | evaluation: |
| | 4 | 301 | 290 | 301 | 0.26 | mild |
| | 5 | 301 | 301 | 301 | 0.00 | irritation |
| | 6 | 301 | 210 | 250 | 3.65 | |

The results show that at the same concentration of mandelic acid, the solution of supramolecular tranexamic acid mandelic acid exhibits no irritation, while the mandelic acid solution has mild irritation. The tests demonstrate that compared to the mandelic acid monomer, the supramolecular tranexamic acid-mandelic acid ionic salt can reduce the irritation of mandelic acid on the skin.

The above-described embodiments are a part but not all of the embodiments of the present application. The detailed description of the embodiments of the present application is not intended to limit the scope of the claimed present application but merely represents selected embodiments of the present application. All other embodiments derived by a person of ordinary skill in the art based on the embodiments of the present application without creative efforts shall fall within the scope of protection of the present application.

1. A supramolecular tranexamic acid-mandelic acid ionic salt, having a structural formula as shown in Formula I:

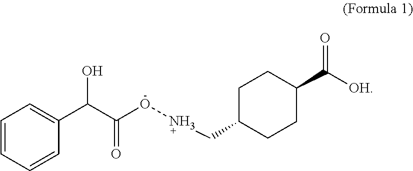

2. The supramolecular tranexamic acid-mandelic acid ionic salt according to claim 1, wherein the supramolecular tranexamic acid-mandelic acid ionic salt comprises a tranexamic acid structure and a mandelic acid structure in a mole ratio of 1:5 to 5:1.

3. The supramolecular tranexamic acid-mandelic acid ionic salt according to claim 1, wherein single crystal data of the supramolecular tranexamic acid-mandelic acid ionic salt is as follows:
chemical formula: C₁₆H₂₃NO₅;
molecular weight: 309.35;
temperature: 149.99 (10) K;
crystal system: orthorhombic;
space group: Pbca;
unit cell parameters: a: 26.126(2)Å, b: 6.2881(5)Å, c: 21.851(2)Å, α: 90°, β: 114.155(11)°, γ: 90°;
volume: 3275.3(6)Å3;
number of units in the unit cell: 8;
calculated density: 1.255 g/cm³;
absorption coefficient: 0.769 mm⁻¹;
F(0000): 1328.0;
crystal size: 0.13 mm×0.11 mm×0.09 mm
radiation: Cu Kα, λ=1.54184;
data collection angle range: 7.416-147.964°;
index range: −32≤h≤27; −7≤k≤6; −27≤1≤26;
collected diffraction points: 16532;
independent diffraction points: 6458, Rint=0.0708, Rsigma=0.0686;
data/limitations/parameters: 6458/0/403;
goodness of fit for F²: 1.062;
final R value, strength>2σ: R₁=0.0719, wR₂=0.1808;
R value of all data: R₁=0.0944, wR₂=0.2072; and
maximum difference peak/hole/: 0.33 e Å⁻³/−0.40 eÅ⁻³.

4. A preparation method for the supramolecular tranexamic acid-mandelic acid ionic salt according to claim 1, wherein the preparation method comprises reacting tranexamic acid and mandelic acid to obtain the supramolecular tranexamic acid-mandelic acid ionic salt as shown in the Formula I.

5. The preparation method according to claim 4, wherein the preparation method comprises:
adding the tranexamic acid and the mandelic acid to an organic solvent and reacting for a predetermined time in a protective gas atmosphere, followed by sonication and stirring to obtain a solution of the supramolecular tranexamic acid-mandelic acid ionic salt; and
crystallizing, filtering, and drying the solution of the supramolecular tranexamic acid-mandelic acid ionic salt to obtain the supramolecular tranexamic acid-mandelic acid ionic salt.

6. The preparation method according to claim 5, wherein the predetermined time is 12 h to 48 h.

7. The preparation method according to claim 5, wherein the organic solvent comprises one or more of acetonitrile, ethanol, and methanol.

8. The preparation method according to claim 5, wherein during the ultrasonic process, at least one of following conditions (a1) to (a5) is satisfied:
(a1) a temperature of an ultrasonic field is 50° C. to 90° C.;
(a2) an ultrasonic frequency is 20 kHz to 60 kHz;
(a3) an ultrasonic power is 700 W to 6000 W;
(a4) an ultrasonic time is 6 h to 12 h; and
(a5) ultrasound for 2 s to 10 s at every interval of 1 s to 5 s.

9. The preparation method according to claim 5, wherein during the stirring process, following conditions (b1) and/or (b2) are satisfied:
(b1) a stirring rate is 30 rad/min to 250 rad/min; and
(b2) a stirring time is 12 h to 48 h.

10. The preparation method according to claim 5, wherein during the drying process, a drying temperature is 50° C. to 90° C.

11. Use of the supramolecular tranexamic acid-mandelic acid ionic salt according to claim 1 as a raw material, in a preparation of pharmaceuticals or cosmetics.

12. The use according to claim 11, wherein the pharmaceuticals or the cosmetics have functions of inhibition of melanocyte activity, inhibition of tyrosinase activity, anti-oxidation, and anti-inflammatory.
